(19) United States
(12) Patent Application Publication
(10) Pub. No.: US 2023/0157949 A1
(43) Pub. Date: May 25, 2023

(54) ORAL FILM OF HIV DRUGS

(71) Applicant: LAURUS LABS LIMITED, Hyderabad (IN)

(72) Inventors: Siva Rama Krishna Velaga, Hyderabad (IN); Siva Ramakrishna RAYALA, Hyderabad (IN); Srikant PIMPLE, Hyderabad (IN); Ananda Haranath UPPALA, Hyderabad (IN); Vamshidhar Reddy DUGGI, Hyderabad (IN); Narendra Kumar KAVURI, Hyderabad (IN)

(21) Appl. No.: 17/920,857

(22) PCT Filed: Jun. 25, 2021
(86) PCT No.: PCT/IB2021/055667

§ 371 Date: Oct. 24, 2022

(30) Foreign Application Priority Data
Jun. 27, 2020 (IN) 202041027365
Oct. 28, 2020 (IN) 202041046918

(51) Int. Cl. A61K 9/00; A61K 31/5365; A61K 31/513; A61K 31/427; A61K 31/52; A61K 31/675; A61K 47/38; A61K 47/36; A61K 47/34; A61K 47/32; A61K 47/10; A61K 47/44

(52) U.S. Cl. CPC A61K 9/006; A61K 31/5365; A61K 31/513; A61K 31/427; A61K 31/52; A61K 31/675; A61K 47/38; A61K 47/36; A61K 47/34; A61K 47/32; A61K 47/10; A61K 47/44

(57) ABSTRACT

The present invention provides oral film of HIV/Anti-retroviral drugs and its pharmaceutically acceptable salt thereof, which is useful for the treatment of HIV infections. Further the present invention provides composition and process for preparing oral film of HIV/Anti-retroviral drugs and its pharmaceutically acceptable salt thereof.

### FIELD OF THE INVENTION

The present invention relates to new oral dosage form of HIV/Anti-retroviral drugs for easy administration. More specifically the present invention relates to oral film of HIV/Anti-retroviral drugs and its pharmaceutically acceptable salt thereof.

### BACKGROUND OF THE INVENTION

HIV (human immunodeficiency virus) is a virus that attacks cells that help the body fight infection, making a person more vulnerable to other infections and diseases. It is spread by contact with certain bodily fluids of a person with HIV, most commonly during unprotected sex (sex without a condom or HIV medicine to prevent or treat HIV), or through sharing injection drug equipment. If left untreated, HIV can lead to the disease AIDS (acquired immunodeficiency syndrome). AIDS is the late stage of HW infection that occurs when the body's immune system is badly damaged because of the virus.

The human body can't get rid of HIV and no effective HIV cure exists. So, once you have HIV, you have it for life. However, by taking HIV medicine (called antiretroviral therapy or ART), people with HIV can live long and healthy lives and prevent transmitting HIV to their sexual partners. In the U.S., most people with HIV do not develop AIDS because taking HIV medicine every day as prescribed stops the progression of the disease.

A person with HIV is considered to have progressed to AIDS when:
the number of their CD4 cells falls below 200 cells per cubic millimeter of blood (200 cells/mm3). (In someone with a healthy immune system, CD4 counts are between 500 and 1,600 cells/mm3.) OR they develop one or more opportunistic infections regardless of their CD4 count.

Without HIV medicine, people with AIDS typically survive about 3 years. Once someone has a dangerous opportunistic illness, life expectancy without treatment falls to about 1 year. HIV medicine can still help people at this stage of HIV infection, and it can even be lifesaving. But people who start ART soon after they get HIV experience more benefits.

Many drugs are approved for HIV therapy and are marketed in various dosage forms like tablets, capsules, oral solution, tablets for oral suspension etc. All of the available dosage forms of oral solution and tablets for suspension are preferably used for pediatric or geriatric patients who cannot swallow tablets or capsule dosage forms. Only few products are available in oral solution and tablets for suspension. Due to unavailability some of the products in oral solution or tablets for suspension, normal tablets/capsules need to be administered either in split tablets or emptying the capsule contents and make dosage as per the requirement.

Dolutegravir is one of the HIV drug recently approved in USA in 2013 and also approved in many countries. It is available under the brand name of TIVICAY® in tablet dosage form of 10mg, 25mg and 50mg strengths. As per the TIVICAY® label treatment for pediatric patients weighing from 30-45kg body weight, the dosage is 35mg once daily (one 25mg tablet and one 10mg tablet) that means they has to take two tablets at a time, which is very difficult for them. TIVICAY® is not approved treatment for pediatric patients weighing below 30kg body weight.

ViiV has recently developed TFOS—Tablets for oral suspension which caters the dosage form for pediatric patients weighing from 3kg and will help to pediatric patients who have difficulty of taking two tablets at a time and unavailability of dosage for less than 30kg body weight. These tablets for oral suspension are approved by USFDA on Jun. 12, 2020 under the brand name of TIVICAY®PD and are available in 5mg strength. Based on the patient body weight TIVICAY®PD 1-6 tablets/day need to be administered. As per TIVICAY®PD label, oral suspension need to be prepared by dispersing the required number of tablets in 5ml/10ml of water in a cup, swirl the contents of cup for 1-2 minutes and its becomes cloudy. This dispersed oral suspension need to be administered within 30 minutes. If any quantity of oral suspension is spilled, then discard the whole suspension and make a new dosage.

Due to extemporaneous preparation of TIVICAY®PD dosage form there will be always dosing errors due to taking more/less no of tablets, some dose may be wasted due to sticking of suspension to the cup or omitting/spilling of the suspension during administration which leads to medication errors and may increase the therapy cost.

Lopinavir is a highly active protease inhibitor but its bioavailability is low and its clearance rapid when given alone. However, in combination with low doses of ritonavir, lopinavir's AUC is increased by greater than 100-fold. Combination product of Lopinavir/Ritonavir is available under the brand name of KALETRA® in tablet dosage form of 200 mg lopinavir/50 mg ritonavir and 100 mg lopinavir/25mg ritonavir, and is also available in Oral Solution containing 400 mg lopinavir and 100 mg ritonavir per 5 mL (80 mg lopinavir and 20 mg ritonavir per mL). As per Prescription KALETRA® label information, before prescribing KALETRA®100/25 mg tablets, children should be assessed for the ability to swallow intact tablets. If a child is unable to reliably swallow a KALETRA® tablet, the KALETRA® oral solution formulation should be prescribed.

KALETRA® oral solution contains 42.4% (v/v) alcohol and 15.3% (w/v) propylene glycol. Even though Lopinavir & Ritonavir is available as Oral Solution, but still has the disadvantages of using solvents like alcohol and propylene glycol which can lead to potential pediatric toxicity, incompatible with polyurethane feeding tubes, less stable and requires special storage condition (to be stored at 2° - 8° C. (36°-46° F.).

The appropriate dose of KALETRA® for each individual pediatric patient is calculated based on body weight (kg) or body surface area (BSA) to avoid under dosing or exceeding the recommended adult dose. Calculation of appropriate dose is cumbersome process and leads to medication errors.

Abacavir and lamivudine are two established components of first-line antiretroviral regimens for the management of HIV infection and the fixed-dose Abacavir/lamivudine formulation has the potential to be an effective, easily adhered to and generally well tolerated component of first-line therapy.

Abacavir sulfate is available as tablets (300 mg) and Oral solution (20 mg/mL) with brand name Ziagen®. Ziagen® Oral Solution is recommended for pediatric patients aged 3months and older and dose is calculated on body weight (kg) and should not exceed 600 mg daily.

Lamivudine is available as tablets (150mg and 300 mg) and Oral solution (10 mg/ml with brand name Epivir®. Oral Solution is recommended for pediatric patients aged 3 months and older and dose is calculated on body weight (kg) and should not exceed 300 mg daily.

The fixed-dose combination of Abacavir and lamivudine was approved in USA in 2004 and also approved in many countries. It is available under the brand name of EPZICOM® in tablet dosage form of 600mg of Abacavir (702 mg of Abacavir sulfate) and 300 mg of Lamivudine as active substances. As per the EPZICOM® label, treatment for pediatric patients weighing at least 25 kg, dosage is one tablet once daily and before prescribing EPZICOM® tablets, paediatric patients should be assessed for the ability to swallow tablets, which is very difficult for patients.

Because EPZICOM® is a fixed-dose tablet and cannot be dose adjusted, EPZICOM® is not recommended in patients requiring dosage adjustment or patients with hepatic impairment and in pediatric patients weighing less than 25 kg. In such cases use of EPIVIR® (lamivudine) oral solution and ZIAGEN® (abacavir) oral solution is considered.

Fixed dose combinations of Abacavir/Dolutegravir/Lamivudine tablets are approved under brand name of TRIUMEQ®. Clinical trials of TRIUMEQ® did not include sufficient numbers of Geriatric patients to determine whether they respond differently from younger subjects. Safety and effectiveness of TRIUMEQ® in pediatric patients have not been established.

Further, many of the cases pediatric/geriatric patients have difficulty in taking dosage forms of oral solution and tablets for oral suspension due to their health condition or taste or flavor.

Once the patient gets HIV infected, the patient need to take medication every day throughout life. Hence, the dosage forms especially for pediatric HIV patients should be more patient compliant so that the dose can be easily administered, palatable, less dosing errors etc.

To overcome dosing errors during tablets for oral suspension preparations, difficulty of swallowing by pediatric/geriatric patients, the present inventors has identified simple solution by developing oral films, which can be administered simply by keeping the film on the tongue, the film disintegrates in less than about 180 seconds. The main advantage of the film over tablets for oral suspension or oral solution is there is no need of water required for dosage form administration, no extemporaneous preparation of the dosage form, no dosing errors, easy administration to all age group of patients, faster absorption etc.

### SUMMARY OF THE INVENTION

In one embodiment, the present invention provides oral film of HIV/Anti-retroviral drugs and its pharmaceutically acceptable salt thereof.

In a preferred embodiment, the present invention provides composition of oral film of HIV/Anti-retroviral drugs and its pharmaceutically acceptable salt thereof, wherein the composition comprises at least one film forming polymer, plasticizer and optionally one or more pharmaceutically acceptable excipients.

In another embodiment, the present invention provides process for preparing oral film comprising one or more HIV/Anti-retroviral drugs and its pharmaceutically acceptable salt thereof.

In another embodiment, the present invention provides process for preparing oral film of HIV/Anti-retroviral drugs and its pharmaceutically acceptable salt thereof, wherein the process may be selected from solvent casting, hot melt extrusion or printing technology.

In another embodiment, the present invention provides oral films of HIV/Anti-retroviral drugs and its pharmaceutically acceptable salt thereof, wherein the film is bioequivalent to existing immediate release oral dosage form that contains the same amount of active pharmaceutical agent.

### DETAILED DESCRIPTION OF THE INVENTION

References in the specification to “one embodiment,” “an embodiment,” “another embodiment,” “a preferred embodiment,” “one aspect,” “another aspect,” “preferred aspect” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described.

As per United States Pharmacopoeia, Films are classified by the site of application. Accordingly, “Oral films” can be formulated to deliver medication to the mouth such as oral hygiene products or to deliver medication to the gastrointestinal tract for absorption. “Buccal films” and “sublingual films” are formulated to facilitate absorption through the proximal mucosal membranes avoiding first pass metabolism or degradation in the gastrointestinal tract and providing a quick onset of action.

“Oral film” or “Oral thin film,” “OTF,” “oral dissolving film,” “oral dissolvable film,” “ODF,” “oral drug strip” or “oral strip” or “oral disintegrating film” according to the present invention refers to a product used to administer active ingredients via absorption in the oral cavity, the stomach (gastrically), and/or via the small intestines (enterically).

The oral film, according to the present invention preferably disintegrates within about three minutes and more preferably within about sixty seconds upon contact with aqueous media or saliva in oral cavity.

An oral film according to the present invention is “non-mucoadhesive” in nature, means that the dosage form is not designed for administration of the active pharmaceutical agent through the oral mucosa i.e. the dosage form is not designed to adhere to the mucosal surfaces of the buccal cavity as an intact film or disintegrated film residue. In particular, the invention provides a non-mucoadhesive orally disintegrating/dissolving film, able to disintegrate upon contact with aqueous media or saliva in oral cavity within about three minutes and more preferably within about sixty seconds.

The term “film” according to the present invention includes films, sheets and wafers, in any size and shape, including rectangular, square, or other desired shape. The films described herein may be any desired thickness and size such that it may be placed into the oral cavity of the user. The films may have a thickness of from about 20 microns to about 300 microns. Films may be in a single layer or they may be multi-layered, such as laminated or co-extruded films.

The term “disintegrating” according to present invention is defined as a state in which any residue of the oral film remaining on the screen of the test apparatus known in the art, or in the mouth, is a soft mass having no palpably film core. The disintegration test does not imply complete solution of dosage unit or even of its active constituent, although a dissolved dosage unit would typically be completely disintegrated. Disintegration of oral film of HIV/Anti-retroviral drugs and its pharmaceutically acceptable salts according to the present invention has its usual and customary meaning in the pharmaceutical arts.

The term “dissolution” according to present invention is defined by the amount of active agent released from the oral film after oral administration or by in-vitro testing known in the art. An in-vitro dissolution test is to evaluate the performance of the product by measuring the amount of active agent dissolved in the dissolution medium. Standardized apparatus known in the art for in-vitro dissolution testing are: USP type I apparatus (basket), USP type II apparatus (Paddle), USP type V apparatus (Paddle over disk).

In one aspect, the present invention provides oral film of HIV/Anti-retroviral drugs and its pharmaceutically acceptable salt thereof.

In another aspect, the present invention provides composition and process for preparing oral film of HIV/Anti-retroviral drugs and its pharmaceutically acceptable salt thereof.

HIV/Anti-retroviral drugs according to the present invention are selected from the group comprising of:
Nucleoside/Nucleotide Reverse Transcriptase Inhibitors (NRTIs) like Abacavir, Didanosine, Emtricitabine, Lamivudine, Entecavir, Stavudine, Tenofovir alafenamide, Tenofovir disoproxil, Zidovudine;
Non-nucleoside Reverse Transcriptase Inhibitors (NNRTIs) like Delavirdine, Doravirine, Efavirenz , Etravirine , Nevirapine , Rilpivirine;
Protease Inhibitors (PIs) like Atazanavir, Amprenavir, Darunavir, Lopinavir, Ritonavir, Fosamprenavir, Indinavir, Nelfinavir, Saquinavir, Tipranavir;
Integrase Inhibitors like Bictegravir, Dolutegravir, Elvitegravir, Cabotegravir, Raltegravir;
Fusion Inhibitors like Enfuvirtide, and
CCR5 Antagonist like Maraviroc and its pharmaceutically acceptable salts thereof.

HIV/Anti-retroviral drugs according to the present invention may also be selected from combination of two or more drugs like Lopinavir/Ritonavir, Dolutegravir/Abacavir/lamivudine, Abacavir/lamivudine, Tenofovir alafenamide/emtricitabine, Dolutegravir/rilpivirine, Dolutegravir/lamivudine, Bictegravir/tenofovir alafenamide/emtricitabine, Elvitegravir/cobicistat/tenofovir alafenamide/emtricitabine , Elvitegravir/cobicistat/tenofovir disoproxil/emtricitabine, Atazanavir/cobicistat, Darunavir/cobicistat, Doravirine/tenofovir disoproxil/lamivudine, Efavirenz/tenofovir disoproxil/emtricitabine, Rilpivirine/tenofovir alafenamide/emtricitabine, Rilpivirine/tenofovir disoproxil/emtricitabine, Abacavir/lamivudine/zidovudine, Tenofovir disoproxil/emtricitabine, Tenofovir disoproxil/lamivudine, Tenofovir alafenamide/lamivudine, Tenofovir disoproxil/emtricitabine/lamivudine, Dolutegravir/lamivudine/tenofovir disoproxil, Dolutegravir/lamivudine/tenofovir alafenamide, Dolutegravir/emtricitabine/tenofovir disoproxil, Dolutegravir/emtricitabine/tenofovir alafenamide, Zidovudine/Lamivudine and its pharmaceutically acceptable salts thereof and any combinations thereof.

Pharmaceutically acceptable salts of HIV/Anti-retroviral drugs according to the present invention is selected from Sodium, Potassium, Magnesium, Hydrochloride, Hydrobromide, Fumarate, Monofumarate, Hemifumarate, Tartrate, Maleate, Succinate, Sulfate, Hemisulfate, Dicarboylic, Glutarate, Benzoate, Carbonate, Mesylate, Besylate or Salicylate.

HIV/Anti-retroviral drugs and its pharmaceutically acceptable salt thereof according to the present invention are may be present in an amount of about 2% to about 75% by weight based on total weight of the composition.

Compositions of oral film according to the present invention comprises one or more pharmaceutically acceptable excipients selected from the group comprising of film forming polymers, plasticizers, suspending/thickening agents, fillers/bulking agents, Disintegrating agents, sweetening agents, taste masking agents, buffering agents, stabilizers, surfactants, anti-foaming agents, flavoring agents and coloring agents.

In one preferred aspect, the present invention provides composition of oral film of HIV/Anti-retroviral drugs and its pharmaceutically acceptable salt thereof, wherein the composition comprises at least one film forming polymer, plasticizer and optionally one or more pharmaceutically acceptable excipients.

In another aspect, the present invention provides oral film of HIV/Anti-retroviral drugs comprises one or more Film forming polymers. The term “Polymers or Film forming polymers” according to the invention is responsible for imparting adequate mechanical properties to films, and they affect the film disintegration and/or dissolution in oral cavity. Film forming polymers used according to the present invention are not limited to hydrophilic or hydrophobic polymers.

Suitable non-limiting hydrophilic film forming polymers according to the present invention are selected from hydroxypropyl methylcellulose or Hypromellose (HPMC), hydroxyethyl cellulose, hydroxypropyl cellulose, sodium carboxy methylcellulose, methyl cellulose, polyvinyl pyrrolidone or povidone, copovidone, polydextrose, polyvinyl alcohol, polyvinyl acetate, polyethylene oxide, pullulan, sodium alginate, propylene glycol alginate, polyacrylic acid, methyl methacrylate copolymer, copolymers of acrylic acid and alkyl acrylate, carboxyvinyl copolymers, modified starch, gelatin, pectin, hydroxypropylethylcellulose, polyoxyethylene stearates, poly-epsilon caprolactone, polyglycolized glycerides, cyclodextrins, galactomannans, Polymerized rosin and combinations thereof. These polymeric materials are freely soluble in aqueous medium and provide the basic component for the film formation.

Suitable non-limiting hydrophobic film forming polymers according to the present invention are selected from ethyl cellulose, Polymethacrylates and combinations thereof.

Film forming polymer/s according to the present invention may be present in an amount of about 1% to about 60% by weight based on total weight of the composition.

In an aspect, the present invention provides oral film of HIV/Anti-retroviral drugs comprises plasticizers. The term “plasticizers” according to the present invention are responsible for mechanical properties of the film, such as tensile strength and decrease the fragility of film by decreasing the glass transition temperature of polymer. Suitable non-limiting plasticizers according to the present invention are selected from polyethylene glycol, propylene glycol, polyethylene-propylene glycol, organic plasticizers with low molecular weights, such as glycerol/glycerin, glycerol monoacetate, diacetate or triacetate, triacetin, polysorbate, cetyl alcohol, propylene glycol, sorbitol, sodium diethylsulfosuccinate, triethyl citrate, tributyl citrate, phthalate derivatives such as dimethyl, diethyl and dibutyl phthalate, citrate derivatives and combinations thereof. Preferable plasticizers according to the present invention are selected from polyethylene glycol, glycerol/glycerin and propylene glycol. Plasticizers according to the present invention may be present in an amount of about 1% to about 30% by weight based on total weight of the composition.

Suspending/thickening agents according to the present invention are responsible for improving the viscosity of the drug and excipient dispersion preparation and consistency of the oral film. Suitable non-limiting suspending/thickening agents according to the present invention are selected from the group comprising of maltodextrin, natural gums like xanthan gum, carrageen, locust bean gum, cellulose derivatives, dextrin, modified starch and combinations thereof. Preferable suspending/thickening agents according to the present invention are selected from Maltodextrin and starches. Suspending/thickening agents according to the present invention may be present in an amount of about 1% to about 50% by weight based on total weight of the composition.

Suitable non-limiting filler/bulking agents according to the present invention are selected from the group comprising of Starches, anhydrous lactose, mannitol, Maltodextrin, dicalcium phosphate, calcium sulfate, cellulose, kaolin, sodium chloride, sorbitol, sucrose and combinations thereof.

Disintegrating agents according to the present invention are responsible for quick disintegration of oral film upon contact with aqueous media or saliva in oral cavity. Suitable non-limiting

Disintegrating agents according to the present invention are selected from the group comprising of Starch, Colloidal silicon dioxide, cellulose derivatives, Crospovidone, Sodium Starch glycolate, Croscarmellose sodium and combinations thereof.

“Sweetening agent or Sweetener” according to the present invention enhances the palatable/taste and pleasurable factor, which makes the film relatively acceptable or agreeable to the patient.

Suitable non-limiting sweeteners according to the present invention are selected from glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Ammonium Glycyrrhizinate, Stevia Rebaudiana (Stevioside); chloro derivatives of sucrose such as sucralose; sugar alcohols such as sorbitol, mannitol, maltodextrin, xylitol, and the like, hydrogenated starch hydrolysates and the synthetic sweetener 3,6-dihydro-6-methyl-1-1-1,2,3-oxathiazin-4-one-2,2-dioxide, particularly the potassium salt (Acesulfame-K), and sodium and calcium salts thereof, natural intensive sweeteners, protein based sweeteners such as thaumatoccous danielli (Thaumatin I and II) and combinations thereof.

“Taste masking agents” according to the present invention are responsible for blocking or diminishing the bitter taste of drug substance or excipients present in the film. Suitable non-limiting taste masking agents according to the present invention are selected from ion exchange resins, cyclodextrins and its derivatives, ready made available mixtures of taste mask enhancers and combinations thereof.

Suitable non-limiting buffering agents according to the present invention are selected from the group comprising of sodium carbonate, sodium bicarbonate, Citric Acid, Sodium Citrate, potassium carbonate, potassium bicarbonate, Ammonium Phosphate Dibasic, Sodium Phosphate

Dibasic, sodium phosphate tribasic, potassium phosphate dibasic, potassium phosphate tribasic, Borate, calcium carbonate, magnesium carbonate, sodium hydroxide, magnesium hydroxide, potassium hydroxide, aluminium hydroxide, and combinations thereof.

Stabilizers according to the present invention are responsible improving the shelf life by preventing the degradation of active agents. Suitable non-limiting stabilizers according to the present invention may be selected from the group comprising of Tocopherol, Ascorbic acid, Citric acid, sodium bisulfite, sodium metabisulfite, propyl gallate, Potassium Metabisulfite, butylated hydroxytoluene and butylated hydroxyanisole and combinations thereof.

Suitable non-limiting surfactants according to the present invention are selected from the group comprising of cetyl alcohol, sodium lauryl sulfate, Polyoxyl 40 Hydrogenated Castor Oil, Spans®, Tweens®, Ethoxylated oils, poloxamers 407 and combinations thereof.

Suitable anti-foaming agents according to the present invention are selected from the group comprising of simethicone or any agent that removes air bubbles/entrapped air/void from film-forming compositions.

“Flavors” used in the present invention are responsible for masking the bitter or nauseating taste of incorporated drug i.e. HIV/anti-retroviral drugs. Suitable non-limiting flavoring agents according to the present invention are selected from the group comprising of natural and synthetic flavoring liquids such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), and combinations thereof.

Suitable coloring agents according to the present invention are selected from the group comprising of food, drug and cosmetic colors (FD&C), drug and cosmetic colors (D&C), or external drug and cosmetic colors (Ext. D&C). These colors are dyes, their corresponding lakes, and certain natural and derived colorants. Other non-limiting examples of coloring agents according to present invention are selected from known azo dyes, organic or inorganic pigments, or coloring agents of natural origin and combinations thereof.

In another aspect, some of the excipients according to the present invention used in the oral film may have one or more functions i.e. an excipient of oral films of the present invention may be multifunctional likely starch used according to present invention may act as a bulking agent and/or disintegrate; Maltodextrin used according to present invention may act as a sweetener and/or suspending/thickening agent and/or filler/bulking agent; Mannitol used according to present invention may act as a sweetener and/or as a filler/bulking agent; cellulose derivatives used according to present invention may act as a film forming polymer and/or a disintegrant and/or suspending/thickening agent. Hence, a skilled person should not construe the limit of an excipient stated or illustrated in any aspect or embodiment or an example of oral film in the present invention to a single function.

In another preferred aspect, the present invention provides composition of oral film of HIV/Anti-retroviral drugs and its pharmaceutically acceptable salt thereof, wherein the composition comprises:
i. from about 2% to about 75% by weight of HIV/Anti-retroviral drugs and its pharmaceutically acceptable salts;
ii. from about 1% to about 60% by weight of at least one film forming polymer;
iii. from about 1% to about 30% by weight of plasticizer; and
iv. optionally one or more pharmaceutically acceptable excipients.

wherein the percentage by weight is relative to the total weight of the composition.

In one aspect, the present invention provides process for preparing oral film of HIV/Anti-retroviral drugs, wherein the process may be selected from the state of the art technologies like solvent casting, hot melt extrusion or printing thereof.

Solvents used according to the present invention in the process for preparing oral dissolvable film of HIV/Anti-retroviral drugs and its pharmaceutically acceptable salts may be selected from water, a polar organic solvent including, but not limited to, ethanol, isopropanol, acetone, methylene chloride, or any combination thereof.

In a preferred aspect, the present invention provides oral films of HIV/Anti retroviral drugs and its pharmaceutically acceptable salts, which are bioequivalent to existing immediate release oral dosage form that contains the same amount of active pharmaceutical agent.

The term “immediate release” according to the invention, refers to a dosage form that allows the drug to dissolve in the gastrointestinal contents, with no intention of delaying or prolonging the dissolution or absorption of the drug. “Immediate release” dosage forms includes tablets, capsules, oral suspensions, oral solutions, orally disintegrating/dispersing tablet (ODT), and other dosage forms intended for immediate release of active ingredient.

In another aspect, the present invention provides oral films of HIV/Anti-retroviral drugs and its pharmaceutically acceptable salts thereof for the treatment of HIV-infection.

The said invention is further illustrated by following non-limiting examples: which are set forth to aid in an understanding of the invention, and are not intended, and should not be construed, to limit in any way the invention set forth in the claims that follow thereafter. A person skilled in the art will readily recognize the various modifications and variations that may be performed without altering the scope of the present invention. Such modifications and variations are encompassed within the scope of the invention and the examples do not in any way limit the scope of the invention.

### EXAMPLE 1

### Unit Compositions of Oral film of Dolutegravir Sodium

| | | Example 1A | Example 1B | Example 1C |
|---|---|---|---|---|
| S. No | Ingredients | (mg) | (mg) | (mg) |
| 1 | Dolutegravir | 10.52 | 10.52 | 10.52 |
| | sodium | | | |
| 2 | Mannitol | 7-10 | 6-8 | — |
| 3 | Hypromellose | 14-18 | 19-23 | 14-18 |
| 4 | Maltodextrin | — | — | 5-11 |
| 5 | Polyvinyl | — | 6-9 | — |
| | Alcohol | | | |
| 6 | Povidone | 5-11 | — | — |
| 7 | Glycerin | 4-8 | 4-8 | 4-8 |
| 8 | Polyethylene | — | 2-4 | — |
| | glycol | | | |
| 9 | Sucralose | 2-4 | 1-3 | 2-4 |
| 10 | Peppermint | 1-2 | 1-2 | 1-2 |
| | Flavor | | | |
| 11 | Orange juice | 3-5 | 3-5 | 3-5 |
| | Flavor | | | |
| 12 | Starch | 0.5-1.5 | — | 1-3 |
| 13 | Purified Water | q.s | q.s | q.s |

| | | | |
|---|---|---|---|
| Weight of Film | 50.0-70.0 | 50.0-70.0 | 50.0-65.0 |

The process used for preparing films of present invention includes solvent casting method and comprises of the following steps

Step 1. Suspension Preparation:

Stage-A: Preparation of polymer dispersion: Add Hydroxypropyl methylcellulose to purified water under continuous stirring to obtain a homogenous dispersion and de-aerate if required.

Stage B: Preparation of Drug solution or dispersion

### Example 1A

Step 1. Add Dolutegravir sodium to purified water under stirring and continue stirring till a homogenous dispersion is obtained.

Step 2. Add povidone to step 1 dispersion under stirring and continue stirring till a homogenous dispersion is obtained.

Step 3. Add Sucralose to step 2 dispersion under stirring and continue stirring till a homogenous dispersion is obtained.

Step 4.Add Mannitol to step 3 dispersion under stirring and continue stirring till a homogenous dispersion is obtained.

Step 5. Add Starch to step 4 under stirring and continue stirring till a homogenous dispersion is obtained.

### Example 1B

Step 1. Add Dolutegravir to purified water under stirring and continue stirring till a homogenous dispersion is obtained.

Step 2. Add polyvinylalcohol to step 1 dispersion under stirring and continue stirring till a homogenous dispersion is obtained.

Step 3. Add Sucralose to step 2 dispersion under stirring and continue stirring till a homogenous dispersion is obtained.

Step 4. Add Mannitol to step 3 dispersion under stirring and continue stirring till a homogenous dispersion is obtained.

### Example 1C

Step 1. Add Dolutegravir to purified water under stirring and continue stirring till a homogenous dispersion is obtained.

Step 2. Add sucralose to step 1 dispersion under stirring and continue stirring till a homogenous dispersion is obtained.

Step 3. Add Maltodextrin to step 2 dispersion under stirring and continue stirring till a homogenous dispersion is obtained.

Step 4. Add Starch to step 3 dispersion under stirring and continue stirring till a homogenous dispersion is obtained.

Stage C: Mix Stage A and B dispersions: Transfer the drug dispersion of stage B to polymeric dispersion of Stage A and mix together to get a uniform dispersion by either stirring or by homogenization.

Stage D: Flavor & Plasticizer Addition: Add the flavor (s) to the dispersion of stage C under stirring, followed by addition of Glycerin and/or add Polyethylene glycol 3350 and continue stirring till a homogenous dispersion is obtained.

Stage E: De-aeration: If any foam is observed, de-aerate the dispersion by applying vacuum under slow stirring.

Step 2. Film Casting: The dispersion of Stage D/stage E is casted using a film casting machine where the dispersion is spreaded on a backing film with a knife to form a thin wet film which is then passed through a drying zone.

Step 3. Slitting: The dried film is cut into a desired size by using slitter.

Step 4. Packing: Pack the film into suitable pouches/sachets.

### EXAMPLE 2

### Oral Film of Lopinavir and Ritonavir

| | | Example 2A | Example 2B |
|---|---|---|---|
| | | Quantity | Quantity |
| | | per unit | per unit |
| S. No. | Ingredients | (mg) | (mg) |
| 1 | Lopinavir | 40.0 | 40.0 |
| 2 | Ritonavir | 10.0 | 10.0 |
| 3 | Copovidone | 40-60 | 40-60 |
| 4 | Polyethylene Glycol | 5-15 | — |
| 5 | Polyoxyl Hydrogenated | — | 5-15 |
| | Castor Oil | | |
| 6 | Taste Enhancer | — | 10-20 |
| 7 | Hypromellose | 40-60 | 30-50 |
| 8 | Glycerine | 15-25 | 10-20 |
| 9 | Acesulfame potassium | 3-8 | 3-8 |
| 10 | Flavor (s) | 2-4 | 2-6 |
| 11 | Ethanol | q.s | q.s |
| 12 | Purified Water | q.s | q.s |
| | Weight of Film | 160.0-220.0 | 150.0-200.0 |

Manufacturing Process:

Step 1. Suspension Preparation:

Stage-A: Preparation of polymer dispersion: Add Hydroxypropyl methylcellulose to purified water under stirring to obtain a homogenous dispersion and de-aerate if required.

Stage B: Preparation of Drug solution or dispersion

### Example 2A

Step 1. Add Lopinavir and Ritonavir to ethanol under stirring and continue stirring till a homogenous dispersion is obtained.

Step 2. Add Copovidone to step 1 dispersion under stirring and continue stirring till a homogenous dispersion is obtained.

Step 3. Add Polyethylene glycol to step 2 dispersion under stirring and continue stirring till a homogenous dispersion is obtained.

### Example 2B

Step 1. Add Lopinavir and Ritonavir to ethanol under stirring and continue stirring till a homogenous dispersion is obtained.

Step 2. Add copovidone to step 1 dispersion under stirring and continue stirring till a homogenous dispersion is obtained.

Step 3. Add Polyoxyl 40 Hydrogenated Castor Oil to step 2 dispersion under stirring and continue stirring till a homogenous dispersion is obtained.

Step 4. Add taste enhancer to step 3 dispersion under stirring and continue stirring till a homogenous dispersion is obtained.

Stage C: Mix of Stage A and Stage B dispersions: Transfer the drug dispersion of stage B to polymeric dispersion of Stage A and mix together to get a uniform dispersion either by stirring or by homogenization.

Stage D: Sweetener, flavor & Plasticizer Addition: Add sweetener (s) to the step C dispersion under stirring, followed by addition of flavor, glycerin and continue stirring till a homogenous dispersion is obtained. Stage E: De-aeration: If any foam observed, de-aerate the dispersion by applying vacuum under slow stirring.

Step 2. Film Casting: The dispersion of Stage D/Stage E is casted using a film casting machine where the dispersion is spreaded on a backing film with a knife to form a thin wet film which is then passed through a drying zone.

Step 3. Slitting: The dried film is cut into a desired size by using slitter.

Step 4. Packing: Pack the film into suitable pouches/sachets.

### EXAMPLE 3

### Oral Film of Abacavir and Lamivudine

| | | Example 3A | Example 3B | Example 3C |
|---|---|---|---|---|
| | | Quantity | Quantity | Quantity |
| | | per unit | per unit | per unit |
| S. No. | Ingredients | (mg) | (mg) | (mg) |
| 1 | Abacavir Sulfate | 35.14 | 35.14 | 35.14 |
| 2 | Lamivudine | 15 | 15 | 15 |
| 3 | Hypromellose | 30-40 | 30-40 | 40-70 |
| 4 | Maltodextrin | 5-10 | 5-10 | 0-10 |
| 5 | Xylitol | — | — | 0-5 |
| 6 | Glycerine | 5-8 | 5-8 | 5-8 |
| 7 | Ammonium | 5-8 | 5-8 | 3-8 |
| | Glycyrrhizinate | | | |
| 8 | Ion exchange resins | — | — | 35-70 |
| 9 | Flavor (s) | 2-4 | 2-4 | 2-4 |
| 10 | Taste enhancer | — | 2-6 | 2-6 |
| 11 | Starch | 2-3 | 2-6 | — |
| 12 | Sodium Citrate | 2-3 | 2-3 | 2-3 |
| 13 | Citric Acid | 1-2 | 1-2 | 1-2 |
| | Purified Water | q.s | q.s | q.s |
| | Weight of Film | 100.0-130.0 | 100.0-130.0 | 140.0-220.0 |

Manufacturing Process:

Step 1. Suspension Preparation:

Stage-A: Preparation of polymer dispersion: Add Hydroxypropyl methylcellulose to purified water under stirring to obtain a homogenous dispersion and de-aerate if required.

Stage B: Preparation of Drug solution or dispersion

### Example 3A

Step 1. Add Abacavir Sulfate to purified water under stirring and continue stirring till a clear solution is obtained.

Step 2. Add Lamivudine to step 1 solution under stirring and continue stirring till a clear solution is obtained.

Step 3. Add Maltodextrin to step 2 solution under stirring and continue stirring till a clear solution is obtained.

Step 4. Add Ammonium Glycyrrhizinate to step 3 solution under stirring and continue stirring till a clear solution is obtained.

Step 5. Add Starch to step 4 solution under stirring and continue stirring till a homogenous dispersion is obtained.

### Example 3B

Step 1. Add Abacavir Sulfate to purified water under stirring and continue stirring till a clear solution is obtained.

Step 2. Add Lamivudine to step 1 solution under stirring and continue stirring till a clear solution is obtained.

Step 3. Add Maltodextrin to step 2 solution under stirring and continue stirring till a clear solution is obtained.

Step 4. Add Ammonium Glycyrrhizinate to step 3 solution under stirring and continue stirring till a clear solution is obtained.

Step 5. Add Starch to step 4 solution under stirring and continue stirring till a homogenous dispersion is obtained.

### Example 3C

Step 1. Add Abacavir Sulfate to purified water under stirring and continue stirring till a clear solution is obtained.

Step 2. Add ion exchange resin to step 1 dispersion under stirring and continue stirring till a homogenous dispersion is obtained and hold the dispersion for about 12 hours.

Step 3. Add Ammonium Glycyrrhizinate to step 2 dispersion under stirring and continue stirring till a homogenous dispersion is obtained.

Step 4. Add Lamivudine to step 3 dispersion under stirring and continue stirring till a homogenous dispersion is obtained.

Step 5. Add Maltodextrin and Xylitol to step 4 dispersion under stirring and continue stirring till a homogenous dispersion is obtained.

Stage C: Mix Stage A and Stage B dispersions: Transfer the drug dispersion of stage B to polymeric dispersion of Stage A and mix together to get a uniform dispersion by either stirring or by homogenization.

Stage D: Flavor & Plasticizer Addition: Add the flavor (s) to the dispersion of stage C under stirring, followed by addition of Glycerine and continue stirring till a homogenous dispersion is obtained.

Stage E: pH adjustment: Add citric acid and sodium citrate to the dispersion of stage D under stirring to adjust the pH and continue stirring till a homogenous dispersion is obtained.

Stage F: De-aeration: If any foam observed, de-aerate the dispersion by applying vacuum under slow stirring.

Step 2 Film Casting: The dispersion is casted using a film casting machine where the dispersion is spreaded on a backing foil with a coating knife to form a thin wet film which is then passed through a drying zone.

Step 3 Slitting: The dried film is cut into a desired size by using slitter.

Step 4 Packing: Pack the film into suitable pouches/sachets.

### EXAMPLE 4

### Oral Film of Abacavir, Dolutegravir and Lamivudine

| | | Example 4A | Example 4B | Example 4C |
|---|---|---|---|---|
| | | Quantity | Quantity | Quantity |
| | | per unit | per unit | per unit |
| S. No. | Ingredients | (mg) | (mg) | (mg) |
| 1 | Abacavir | 70.28 | 70.28 | 70.28 |
| | Sulfate | | | |
| 2 | Dolutegravir | 5.26 | 5.26 | 5.26 |
| | Sodium | | | |
| 3 | Lamivudine | 30 | 30 | 30 |
| 4 | Hypromellose | 60-80 | 60-80 | 60-80 |
| 5 | Maltodextrin | 10-15 | 10-15 | 0-10 |
| 6 | Xylitol | — | — | 0-10 |
| 7 | Glycerin | 10-20 | 10-15 | 10-15 |
| 8 | Sucralose | 10-15 | 10-15 | 4-10 |
| 9 | Ion exchange | — | — | 70-120 |
| | resins | | | |
| 10 | Flavor (s) | 3-6 | 3-6 | 3-6 |
| 11 | Taste enhancer | — | 4-10 | 4-10 |
| 12 | Sodium Citrate | 4-6 | 4-6 | 3-6 |
| 13 | Citric Acid | 2-4 | 2-4 | 2-3 |
| | Purified Water | q.s | q.s | q.s |
| | Weight of Film | 210.0-250.0 | 210.0-250.0 | 250.0-320.0 |

Manufacturing Process

Step 1. Suspension Preparation:

Stage-A: Preparation of polymer dispersion: Add Hydroxypropyl methylcellulose to purified water under stirring to obtain a homogenous dispersion and de-aerate if required.

Stage B: Preparation of Drug solution or dispersion

### Example 4A

Step 1. Add Abacavir Sulfate to purified water under stirring and continue stirring till a clear solution is obtained.

Step 2. Add Lamivudine to step 1 solution under stirring and continue stirring till a clear solution is obtained.

Step 3. Add Dolutegravir sodium to step 2 solution under stirring and continue stirring till a homogenous dispersion is obtained.

Step 4. Add Maltodextrin to step 3 dispersion under stirring and continue stirring till a homogenous dispersion is obtained.

Step 5. Add Sucralose to step 4 solution under stirring and continue stirring till a homogenous dispersion is obtained.

### Example 4B

Step 1. Add Abacavir Sulfate to purified water under stirring and continue stirring till a clear solution is obtained.

Step 2. Add Lamivudine to step 1 solution under stirring and continue stirring till a clear solution is obtained.

Step 3. Add Dolutegravir sodium to step 2 solution under stirring and continue stirring till a homogenous solution is obtained.

Step 4. Add Maltodextrin to step 3 solution under stirring and continue stirring till a homogenous dispersion is obtained.

Step 5. Add Sucralose to step 4 solution under stirring and continue stirring till a clear solution is obtained.

### Example 4C:

Step 1. Add Abacavir Sulfate to purified water under stirring and continue stirring till a clear solution is obtained.

Step 2. Add ion exchange resin to step 1 dispersion under stirring and continue stirring till a homogenous dispersion is obtained and hold the dispersion for about 12 hours.

Step 3. Add Masking flavor to step 2 dispersion under stirring and continue stirring till a homogenous dispersion is obtained

Step 4. Add Sucralose to step 3 dispersion under stirring and continue stirring till a homogenous dispersion is obtained.

Step 5. Add Lamivudine to step 4 dispersion under stirring and continue stirring till a homogenous dispersion is obtained.

Step 6. Add Dolutegravir sodium to step 5 dispersion under stirring and continue stirring till a homogenous dispersion is obtained.

Step 7. Add Maltodextrin and/or Xylitol to step 6 dispersion under stirring and continue stirring till a homogenous dispersion is obtained.

Stage C: Mix of Stage A and B dispersions: Transfer drug dispersion of stage B to polymeric dispersion of Stage A and mix together to get a uniform dispersion by either stirring or by homogenization.

Stage D: Taste enhance, Flavor & Plasticizer Addition: Add the flavor (s) to the dispersion of stage

C under stirring, followed by addition of Glycerine and continue stirring till a homogenous dispersion is obtained.

Stage E: pH adjustment: Add citric acid and sodium citrate to the dispersion of stage D under stirring and continue stirring till a homogenous dispersion is obtained.

Stage F: De-aeration: If any foam observed, de-aerate the dispersion by applying vacuum under slow stirring.

Step 2. Film Casting: The dispersion is casted using a film casting machine where the dispersion is spreaded on a backing foil with a coating knife to form a thin wet film which is then passed through a drying zone.

Step 3. Slitting: The dried film is cut into a desired size by using slitter.

Step 4. Packing: Pack the film into suitable pouches/sachets.

### EXAMPLE 5

### Dolutegravir Oral Film 10 mg

| | | Unit compositions of oral film of Dolutegravir 10 mg | | |
|---|---|---|---|---|
| | | Example 5A | Example 5B | Example 5C |
| S. No | Ingredient | (mg/Film) | (mg/Film) | (mg/Film) |
| 1. | Dolutegravir Sodium | 10.52 | 10.52 | 10.52 |
| 2. | Hypromellose | 16.00 | 16.00 | 16.00 |
| 3. | Maltodextrin | 5.28 | — | 5.28 |
| 4. | Povidone | — | 5.00 | — |
| 5. | Starch | — | 2.00 | 2.00 |
| 6. | Glycerine | 5.00 | 5.00 | 5.00 |
| 7. | Sucralose | 3.20 | 3.48 | 3.20 |
| 8. | Orange Flavour | 2.00 | 2.00 | 2.00 |
| 9. | Peppermint Flavour | 1.00 | 1.00 | 1.00 |
| 10. | Purified Water | q.s | q.s | q.s |
| | Film Weight | 43.00 | 45.00 | 45.00 |

Brief process:

Stage A: Hypromellose was added to purified water and stirred till uniform dispersion was observed.

Stage B: Dolutegravir sodium was added slowly to stage A dispersion under stirring and stirring was continued till uniform dispersion was observed. Materials like Maltodextrin/Povidone/starch and Sucralose were added to suspension under stirring and stirring was continued till uniform dispersion was observed.

Stage C: Glycerin was added to stage B suspension under stirring and stirring was continued till uniform dispersion was observed.

Stage D: Orange flavor and Peppermint flavor were added to stage C suspension under stirring and stirring was continued till uniform dispersion was observed.

Stage E: Dispersion of step D was subjected to homogenization till a homogenous dispersion was observed and de-aerate the dispersion by applying vacuum under slow stirring.

Film Casting: The dispersion of stage E is casted using a film casting machine where the dispersion is spreaded on a backing film with a knife to form a thin wet film which is then passed through a drying zone.

Slitting: The dried film is cut into a desired size by using slitter.

Packing: Pack the film into suitable pouches/sachets

Dissolution Profile: Dissolution of the oral films is conducted and compared with that of Reference Product TIVICAY®PD (Dolutegravir) TFOS (Tablets for oral suspension) 5mg. The results of the same are given in below table:

Dissolution Conditions: 0.01M pH 6.8 Phosphate buffer
media, 900 mL, USP Type-I Basket, 100 rpm

| | TIVICAY ®PD | | | |
|---|---|---|---|---|
| | TFOS | Oral film of Dolutegravir 10 mg | | |
| | (Dolutegravir) | Example - | Example - | Example - |
| Time | 5 mg | 5A | 5B | 5C |
| Minutes | % Mean (Cumulative % drug dissolved) | | | |
| 5 | 39 | 92 | 89 | 81 |
| 10 | 50 | 93 | 92 | 87 |
| 15 | 57 | 94 | 93 | 89 |
| 20 | 63 | 96 | 95 | 92 |
| 30 | 68 | 96 | 95 | 94 |
| 45 | 74 | 97 | 96 | 96 |
| Infinity | 80 | 98 | 97 | 97 |

Bioequivalence Testing

Bioequivalence testing typically requires an in-vivo test in healthy human volunteers in which the concentration of the active ingredient or active moiety, and, when appropriate, its active metabolite(s), in whole blood, plasma, serum, or other appropriate biological fluid is measured as a function of time.

A standard in-vivo BE study design is based on the administration of either single or multiple doses of the test and reference products to healthy subjects on separate occasions, with random assignment to the two possible sequences of drug product administration. Statistical analysis for pharmacokinetic measures, such as area under the curve (AUC) and peak concentration (Cₘₐₓ), is preferably based on the so-called “two one-sided tests procedure” to determine whether the average values for the pharmacokinetic measures determined after administration of the test and reference products are comparable.

The oral film of Dolutegravir 10 mg from ‘Example 5C’ and Reference product Tivicay® PD (Dolutegravir) TFOS 5 mg (two tablets) are studied for Bioequivalence in healthy human subjects in a randomized, single dose, two way, two sequence crossover, open label with seven days washout period study under fasting conditions. The results (Pharmacokinetic (PK) parameters) from Bioequivalence study are given below:

| | Least square Geometric | | | | |
|---|---|---|---|---|---|
| | Means | | | | |
| | Example 5C | Reference | | | |
| | (Oral film of | (Tivicay ® PD | | 90% Confidence | |
| | Dolutegravir | TFOS 5 mg | T/R | Intervals | |
| PK Parameters | 10 mg) | (two tablets)) | Ratio | Lower | Upper |
| Cₘₐₓ | 1063.2629 | 1121.0167 | 94.85 | 88.99 | 101.09 |
| (ng/ml) | | | | | |
| AUC₀₋ₜ | 17854.1274 | 18691.3582 | 95.52 | 90.05 | 101.33 |
| (hr*ng/ml) | | | | | |
| AUC_{0-inf} | 19107.1275 | 19828.2805 | 96.36 | 90.91 | 102.15 |
| (hr*ng/ml) | | | | | |

From the comparable PK measures, the oral film of Dolutegravir 10mg according to the invention is bioequivalent to reference product—Tivicay® PD TFOS (Tablets for oral suspension) 5mg (two tablets).

### EXAMPLE 6

### Abacavir and Lamivudine Oral Films 30 mg/15mg:

| | Ingredients | Example 6A | Example 6B | Example 6C |
|---|---|---|---|---|
| S. No. | Strategy | (mg/film) | (mg/film) | (mg/film) |
| 1. | Abacavir base | 30.00 | 30.00 | 30.00 |
| 2. | Lamivudine | 15.00 | 15.00 | 15.00 |
| 3. | Hypromellose | 47.55 | 47.55 | 47.50 |
| 4. | Sucralose | 3.00 | 3.00 | 3.00 |
| 5. | Maltodextrin | 8.00 | 8.00 | 7.79 |
| 6. | Glycerol | 12.00 | 12.00 | 11.00 |
| 7. | Sodium Saccharin | 0.45 | 0.45 | 0.45 |
| 8. | Acesulfame | 5.00 | 5.00 | 5.00 |
| | Potassium | | | |
| 9. | Starch | — | — | 5.00 |
| 10. | Butylated Hydroxy | — | 0.08 | 0.13 |
| | Anisole | | | |
| 11. | Butylated Hydroxy | — | 0.08 | 0.13 |
| | Toluene | | | |
| 12. | Strawberry Flavor | 3.00 | 3.00 | 3.00 |
| 13. | Banana Flavor | 3.00 | 3.00 | 2.00 |
| 14. | Purified Water | Q.s | Q.s | Q.S |
| 15. | Ethanol | — | Q.s | Q.s |

| | | | |
|---|---|---|---|
| Total Film Wt. (mg) | 127.00 | 127.16 | 130.00 |

Brief manufacturing procedure

Step 1: Purified water used for the batch should be purged with Nitrogen.

Step 2: Lamivudine was added to part quantity of purified water under continuous Stirring and continue stirring to clear solution is obtained

Step 3: Abacavir was added to remaining quantity of purified water under stirring and stirring was continued till uniform dispersion was observed.

Step 4: Step 3 Abacavir dispersion was homogenized.

Step 5: Mix Lamivudine solution of step 2 and Abacavir dispersion of step 4 using suitable stirrer. Followed by add materials like Glycerol, Hypromellose, Sucralose, Sodium Saccharin, Acesulfame Potassium, Maltodextrin and Starch were added one after another to suspension under stirring and stirring was continued till uniform dispersion was observed.

Step 6: Dissolve Butylated Hydroxy Toluene and Butylated Hydroxy Anisole in ethanol and add to dispersion of step 5 under stirring and add flavors to the same under stirring and stirring was continued till uniform dispersion was observed.

Step 7: Homogenize the dispersion obtained from step 8 followed by nitrogen purging.

Step 8: Cast the suspension obtained from Step 9 using film casting machine.

Dissolution Profile:

Dissolution Condition: 0.1N HCl, 900
mL, USP Type-I Basket, 100 rpm

| | Formulation | | |
|---|---|---|---|
| | Example- 6A | Example-6B | Example - 6C |
| Time (min) | % Mean | % Mean | % Mean |

Abacavir (Cumulative % drug dissolved)

| | | | |
|---|---|---|---|
| 5 | 67 | 68 | 90 |
| 10 | 88 | 87 | 95 |
| 15 | 93 | 94 | 96 |
| 20 | 95 | 97 | 96 |
| 30 | 97 | 99 | 97 |

Lamivudine (Cumulative % drug dissolved)

| | | | |
|---|---|---|---|
| 5 | 79 | 78 | 97 |
| 10 | 95 | 92 | 100 |
| 15 | 99 | 98 | 101 |
| 20 | 100 | 100 | 101 |
| 30 | 101 | 102 | 102 |

1. An oral film of HIV/Anti-retroviral drugs, wherein the oral film comprises:
a. an HIV/Anti-retroviral drug selected from the group consisting of Abacavir, Emtricitabine, Lamivudine, Zidovudine, Efavirenz, Bictegravir, Dolutegravir, Elvitegravir, Cabotegravir, Rilpivirine, Raltegravir, Atazanavir, Darunavir, Lopinavir, or Ritonavir or combinations of HIV/Anti-retroviral drugs selected from the group consisting of Lopinavir/Ritonavir, Dolutegravir/Abacavir/Lamivudine, Abacavir/Lamivudine, Tenofovir al afenamide/Emtricitabine, Dolutegravir/Rilpivirine, Dolutegravir/Lamivudine, Bictegravir/Tenofovir al afenami de/Emtricitabine, Efavirenz/Tenofovir disoproxil/Emtricitabine, Rilpivirine/tenofovir alafenamide/emtricitabine, Rilpivirine/Tenofovir disoproxil/Emtricitabine, Abacavir/lamivudine/zidovudine, Tenofovir disoproxil/emtricitabine, Tenofovir disoproxil/lamivudine, Tenofovir alafenamide/lamivudine, Tenofovir di soproxil/emtri citabine/lamivudine, Dolutegravir/lamivudine/tenofovir disoproxil, Dolutegravir/lamivudine/tenofovir alafenamide, Dolutegravir/emtricitabine/tenofovir di soproxil, Dolutegravir/emtricitabine/tenofovir alafenamide, or Zidovudine/Lamivudine, or their pharmaceutically acceptable salts thereof,
b. at least one film forming polymer, and
c. at least one plasticizer.

2. (canceled)

3. (canceled)

4. The oral film of HIV/Anti-retroviral drugs as claimed in claim 1, wherein the at least one film forming polymer is selected from the group consisting of hydroxypropyl methylcellulose, hydroxyethyl cellulose, hydroxypropyl cellulose, sodium carboxy methylcellulose, methyl cellulose, polyvinyl pyrrolidone or povidone, copovidone, polydextrose, polyvinyl alcohol, polyvinyl acetate, polyethylene oxide, pullulan, sodium alginate, propylene glycol alginate, polyacrylic acid, methyl methacrylate copolymer, copolymers of acrylic acid and alkyl acrylate, carboxyvinyl copolymers, modified starch, gelatin, pectin, hydroxypropylethylcellulose, polyoxyethylene stearates, poly-epsilon caprolactone, polyglycolized glycerides, cyclodextrins, galactomannans, Polymerized rosin ethyl cellulose, Polymethacrylates and combinations thereof.

5. The oral film of HIV/Anti-retroviral drugs as claimed in claim 1, wherein the at least one plasticizer is selected from the group consisting of polyethylene glycol, a polyethylene-propylene glycol, glycerol/glycerin, glycerol monoacetate, glycerol diacetate, glycerol triacetate, triacetin, polysorbate, cetyl alcohol, propylene glycol, sorbitol, sodium diethylsulfosuccinate, triethyl citrate, tributyl citrate, dimethyl phthalate, diethyl phthalate, dibutyl phthalate and combinations thereof.

6. The oral film of HIV/Anti-retroviral drugs as claimed in claim 1, further comprising one or more pharmaceutically acceptable excipients selected from the group consisting of sweetening agents, taste masking agents, buffering agents, suspending/thickening agents, disintegrating agents, fillers/bulking agents, stabilizers, surfactants, anti-foaming agents, flavoring agents, and coloring agents.

7. (canceled)

8. (cancelled)

9. An oral film of Dolutegravir comprising:
a. Dolutegravir or its pharmaceutically acceptable salt,
b. at least one film forming polymer, and
c. at least one plasticizer.

10. An oral film of Lopinavir and Ritonavir comprising:
a. Lopinavir or its pharmaceutically acceptable salt,
b. Ritonavir or its pharmaceutically acceptable salt,
c. at least one film forming polymer, and
d. at least one plasticizer.

11. An oral film of Abacavir and Lamivudine comprising:
a. Abacavir or its pharmaceutically acceptable salt,
b. Lamivudine or its pharmaceutically acceptable salt,
c. at least one film forming polymer, and
d. at least one plasticizer.

12. An oral film of Abacavir, Dolutegravir, and Lamivudine comprising:
a. Abacavir or its pharmaceutically acceptable salt,
b. Dolutegravir or its pharmaceutically acceptable salt,
c. Lamivudine or its pharmaceutically acceptable salt,
d. at least one film forming polymer, and
e. at least one plasticizer.

13. An oral film of Emtricitabine and Tenofovir alafenamide comprising:
a. Emtricitabine or its pharmaceutically acceptable salt,
b. Tenofovir alafenamide or its pharmaceutically acceptable salt,
c. at least one film forming polymer, and
d. at least one plasticizer.
